(19) United States
(12) Patent Application Publication
(10) Pub. No.: US 2009/0157076 A1
(43) Pub. Date: Jun. 18, 2009

(54) DEVICES AND SYSTEMS FOR MINIMALLY INVASIVE SURGICAL PROCEDURES

(76) Inventors: William L. Athas, Chapel Hill, NC (US); Thomas B. Miller, Perkiomenville, PA (US); Aurora Pryor, Durham, NC (US); Geoffrey A. Orth, Sebastopol, CA (US); Michael S. Williams, Santa Rosa, CA (US); Jeffrey A. Smith, Petaluma, CA (US); Richard A. Glenn, Santa Rosa, CA (US); Daniel W. Fifer, Windsor, CA (US)

Correspondence Address: STALLMAN & POLLOCK LLP, P.O. Box 26450, SAN FRANCISCO, CA, 94126, US

(21) Appl. No.: 12/209,586

(22) Filed: Sep. 12, 2008

Related U.S. Application Data
(60) Provisional application No. 60/971,900, filed on Sep. 12, 2007.

(51) Int. Cl. A61B 17/00; A61B 17/12; A61B 18/18

(52) U.S. Cl. 606/41; 606/190; 606/139

(57) ABSTRACT

A system for minimally invasive medical procedures includes an elongate tubular access cannula comprising an elongate tubular member having a rigid proximal portion and an articulating portion. The tubular member has a first branch and a tubular bifurcation extending from the first branch, allowing simultaneous use of multiple instruments. A dissector suitable for use with the access cannula, or other access devices, for implantation of gastric bands or for other procedures includes a pre-curved distal portion having a dissection element such as a monopolar RF conductor and/or a dissection balloon, as well as a snare. In one method, the dissector is advanced around the posterior side of the stomach to form a tunnel in the connective tissue, and the snare is then extended from the dissector to engage a portion of the band and withdraw it through the tunnel.

### RELATED APPLICATIONS

This application claims the benefit of U.S. Provisional Application No. 60/971,900, filed Sep. 12, 2007, Attorney Docket No. TRX-1100, which is incorporated herein by reference.

### TECHNICAL FIELD OF THE INVENTION

The present invention relates to the field of systems for performing surgical procedures through minimally invasive access ports.

### BACKGROUND

Surgery in the abdominal cavity is typically performed using open surgical techniques or laparoscopic procedures. Each of these procedures requires incisions through the skin and underlying muscle and peritoneal tissue, and thus results in the potential for post-surgical scarring and/or hernias. Laparoscopic procedures, while less invasive than open surgical techniques, require multiple small incisions or ports to gain access to the peritoneal site using the various instruments and scopes needed to complete the procedure. Further developments have lead to systems allowing procedures to be performed using only a single port.

Systems and techniques in which access to the abdominal cavity is gained through a natural orifice (so-called “NOTES” procedures) are advantageous in that incisions through the skin and underlying muscle and peritoneal tissue may be avoided. Use of such systems can provide access to the peritoneal cavity using an access device inserted into the esophagus, stomach or intestine (via, for example, the mouth, vagina, or rectum). Instruments are then advanced through the access device into the peritoneal cavity via an incision in the wall of the esophagus, stomach or intestine. The present application describes an articulating cannula suitable for use in single port surgery (“SPS”) and NOTES procedures.

### BRIEF DESCRIPTION OF THE DRAWINGS

FIG. 1A is a perspective view of an articulating cannula.

FIG. 1B is a side cross-section view of the cannula of FIG. 1A.

FIG. 1C is a cross-section view of the cannula taken along the plane designated 1C-1C in FIG. 1B.

FIG. 2 is a top cross-section view of the cannula of FIG. 1A.

FIG. 3 is a side elevation view of a portion of the cannula of FIG. 1A, showing the cannula distal portion in a neutral position, and further showing in dashed lines two articulated positions for the distal portion.

FIG. 4A is a side elevation view of a portion of an alternative cannula having a bifurcated distal portion.

FIGS. 4B and 4C show the cannula of FIG. 4A in articulated positions.

FIG. 5A is a side perspective view of an alternative cannula including finger retractors.

FIG. 5B is a side perspective view of an alternative cannula including a conical retractor.

FIGS. 6-21 are a series of drawings schematically illustrating use of the cannula for positioning an implant within the abdominal cavity during a single port procedure.

FIGS. 22A-22D illustrate a second example of an implantation method.

FIGS. 23A-23D illustrate dissectors that may be used in the method of FIGS. 6-21.

FIG. 24 is a perspective view of an alternative dissector that further includes a snare.

FIG. 25 is a cross-section view taken along the plane designated 25-25 in FIG. 24.

FIG. 26 is a perspective view of the distal portion of the dissector of FIG. 24, showing the dissection wire in a deployed position. The optional balloon is not shown.

FIG. 27 is a perspective view similar to FIG. 26, showing the snare deployed.

### DETAILED DESCRIPTION

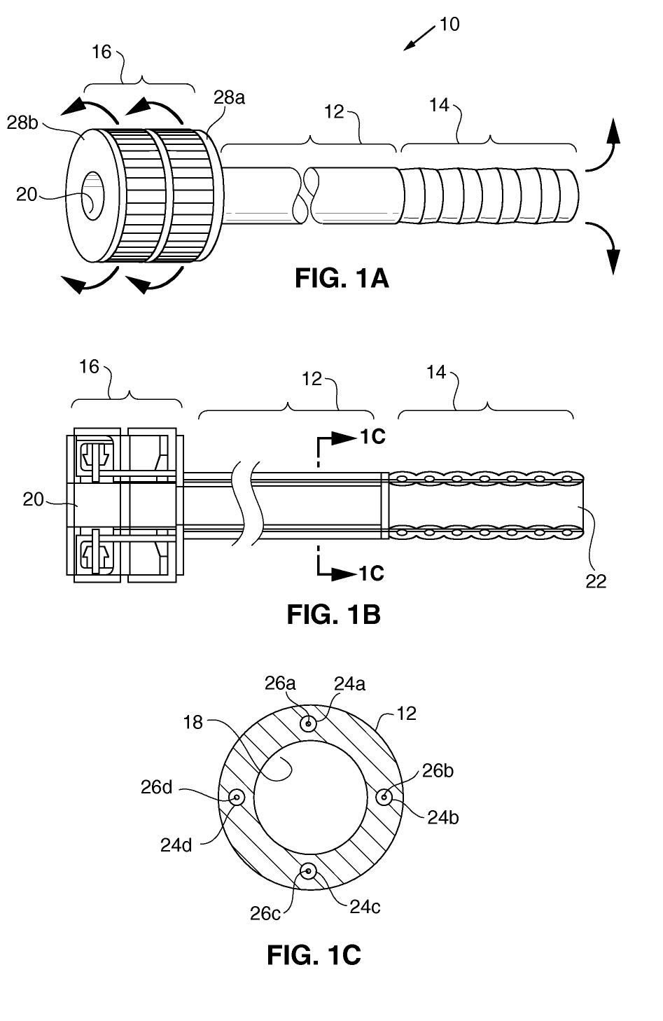

FIGS. 1A and 1B show an exemplary body of an articulating cannula 10. Cannula 10 includes a proximal section 12 of fixed orientation and an articulatable distal section 14. Controls 16 at the proximal end allow the user to control articulation of the distal section 14. An instrument channel 18 extends through the access cannula 10. The instrument channel 18 receives instruments via an instrument port 20 positioned at the proximal end of the cannula 10 and may be proportioned to received multiple instruments at one time. The instruments may be extended from the cannula 10 into a body cavity via an exit port 22 at the proximal end of the cannula. The cannula is constructed to maintain its desired shape under the stresses imparted to it during the use of instruments accessing an operative site through the instrument channel.

Referring to FIG. 1C, fixed section 12 is formed of a length of tubing having the instrument channel 18 extending through it. Pullwire lumens 24a-d extend through the walls of the fixed section 12. Pullwires 26a-d extend through these lumens 24a-d and are anchored within the distal section 14. The numbers and positions of the pullwires and associated lumens are selected based upon the articulation requirements for the distal section 14. In the illustrated embodiment, four pullwires such are positioned at 90 degree intervals allowing for up-down and left-right articulation, although alternative pullwire quantities and arrangements may instead be used.

Articulatable distal section 14 may be formed of a length of tubing or a plurality of spine elements strung together over the pullwires. The distal portion may include rigidizing or “shape lock” elements allowing the distal portion of the cannula to be selectively rigidized at a desired curvature.

Activation of the pullwires is achieved using control knobs 28a, 28b, each of which is independently rotatable about the longitudinal axis of the cannula 10. In the illustrated embodiment, knob 28a drives a gear system that applies and releases tension on pullwires 26b, 26d (FIG. 1C) so as to cause lateral (left-right) articulation of the cannula 10. In a similar way, knob 28b actuates pullwires 26a, 26c for upward-downward articulation of the cannula 10.

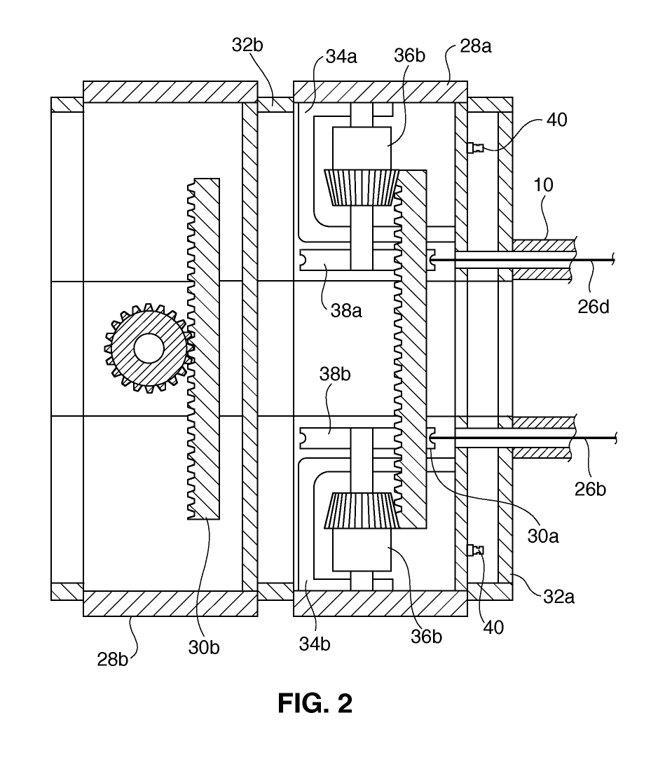

FIG. 2 illustrates details of a gear system that may be used for pullwire activation. As shown, knob 28a includes a ring gear 30a such that rotation of the knob 28a likewise rotates the ring gear 30a. Ring gear 30a includes distally-oriented teeth as shown.

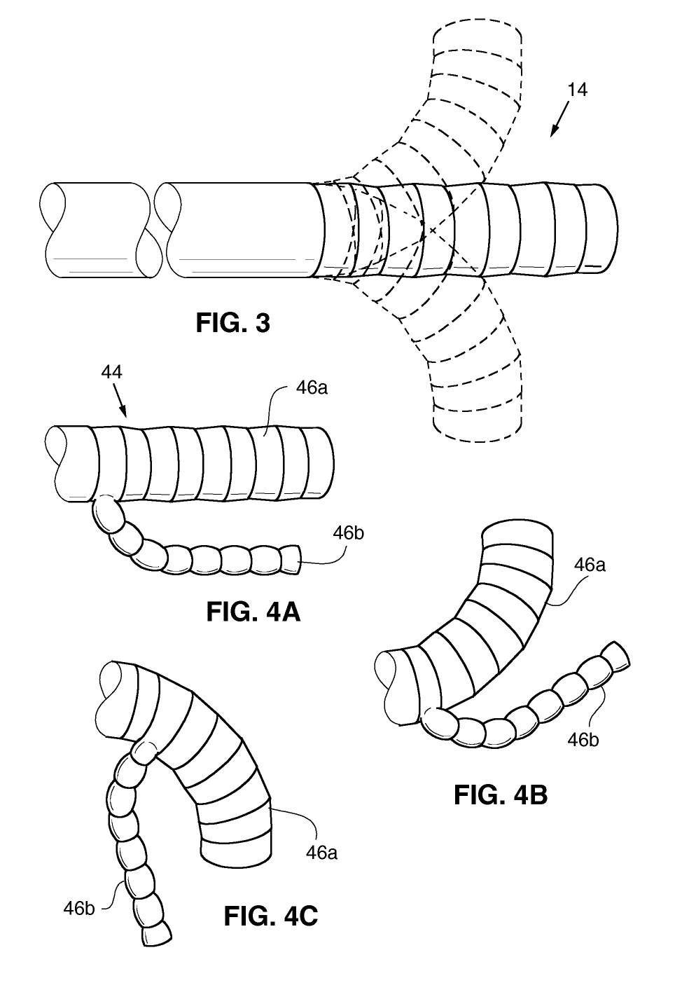

A collar 32a is fixed about the shaft of the cannula 10. A pair of brackets 34a,b are attached to the collar 32a, and each bracket 34a,b supports a beveled spur gear 36a,b having teeth in engagement with the ring gear 30a as shown. Each spur gear 36a,b drives a corresponding pulley 38a,b. Pullwire 26b is coupled to pulley 38b, and pullwire 26d is coupled to pulley 38a. Thus, rotation of each spur gear 36a will rotate its corresponding pulley, causing the associated pullwire to either be drawn around the pulley (thereby causing articulation of the cannula in the direction associated with that pullwire), or to pay out from the pulley. In the particular system shown in FIG. 2, the arrangement of gears is such that rotation of the control knob 28a in a clockwise direction (relative to the user) will deflect the distal cannula portion 14 towards the right, and rotation of the knob 28a in a counterclockwise direction will deflect the cannula portion 14 to the left. FIG. 3 illustrates left, right, and neutral (unarticulated) positions for the distal cannula portion 14.

The controls 16 may include a locking feature that allows the articulated position of the distal cannula portion 14 to be temporarily fixed. For example, a plurality of spring detents 40 on a distal-facing surface of knob 28a are positioned to snap into engagement with corresponding catches 42 on the proximal-facing surface of collar 32a to lock the position of knob 28a. Multiple such catches 42 are included so as to allow the left-right cannula articulation to be locked at any desired position.

A preferred embodiment performs upward-downward deflection using a system having features that are like those described above, but offset 90 degrees from those used for left-right articulation. For upward-downward deflection, knob 28a includes a ring gear 30b. Collar 32b on the cannula 10 supports bevel spur gears, pulleys and associated components that actuate the pullwires 26a, 26c (FIG. 1C). Because these components are the same as those used for left-right deflection, a detailed discussion is not provided.

FIG. 4A shows an alternate embodiment of a cannula distal portion 44 having a bifurcated configuration. Cannula distal portion 44 includes one or more tubular branches 46a, 46b which may be of equal or differing sizes and which may be symmetrically or asymmetrically arranged. In the illustrated embodiment, branch 46a provides the cannula with a main lumen and is articulatable using a system similar to that described above. Branch 46b is shown as a smaller diameter tube branching off of the main branch 46a for use in supporting an endoscope or other instrument. Branch 46b may optionally be an articulatable branch, or it might be arranged such that it will articulate with the main branch 46a as illustrated in FIGS. 4B and 4C. In other words, as main branch 46a is articulated upwardly or to the left as shown in FIG. 4B, it will cause side branch 46b to move to a similar orientation such that both branches remain oriented towards a target surgical site.

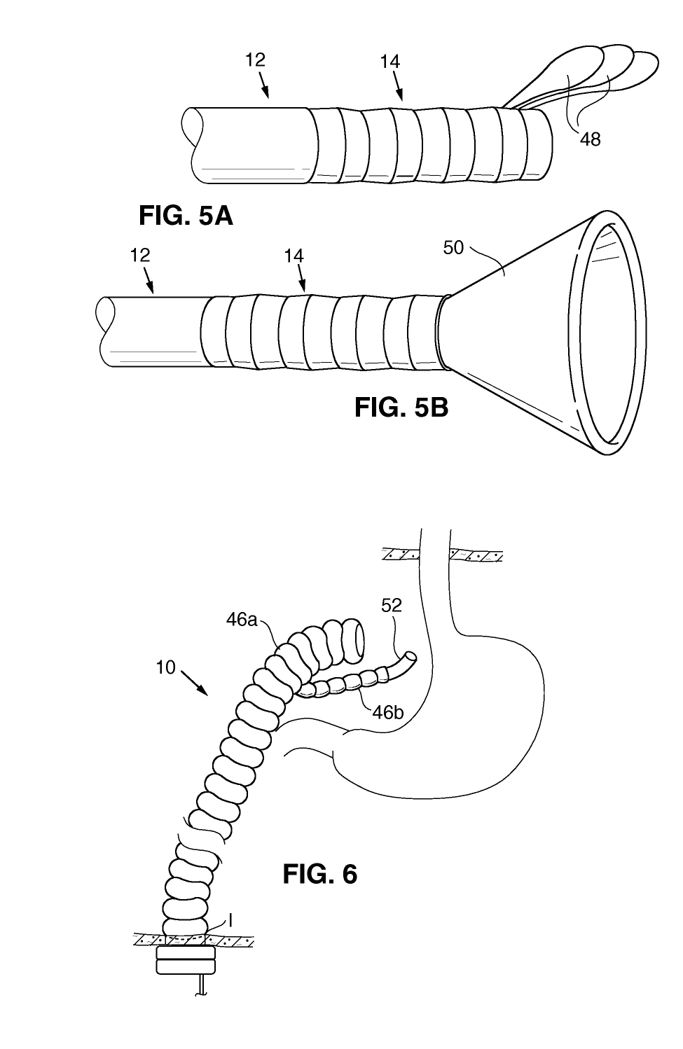

In a further modification shown in FIGS. 5A and 5B, mechanically or pneumatically deployed retractor elements may be positioned on the distal end of the cannula so as to maintain a working space surrounding the cannula distal portion 14. As but two examples of the various configurations that might be used, the retractor elements might include one or more fingers 48 (FIG. 5A) or a hollow cone 50 that flares from the distal end of the cannula.

The system illustrated in the accompanying drawings allows surgical procedures to be carried out through a single port formed in an abdominal wall. The port may be formed using conventional techniques in a chosen location, or it may be formed through the umbilicus. In alternate embodiments, the cannula may be use to gain access to a body cavity of a patient via a natural orifice (e.g. mouth, rectum, vaginal opening) into a hollow organ (esophagus, stomach, intestine, vagina or uterus).

FIGS. 6 through 21 schematically illustrate use of the bifurcated cannula of FIG. 4A to position a medical implant surrounding the stomach. This procedure might be used to place a gastric band (e.g. Lap-Band or Swedish Band) of the type known in the art, or to place more recently developed devices, including those disclosed in U.S. application Ser. No. ______ entitled “Satiation Devices and Methods for Controlling Obesity”, filed July, 2008, (based on U.S. Provisional Application No. 60/958,122, filed Jul. 3, 2007) and U.S. application Ser. No. ______, entitled “Devices for Treating Gastroesophageal Reflux Disease and Hiatal Hernia and Methods for Treating Gastroesophageal Reflux Disease and Hiatal Hernia using Same”, filed Jul. ______, 2008, (based on U.S. Provisional Application No. 60/958,303 filed Jul. 3, 2007), both of which are assigned to the assignee of the present invention. Although the procedure as illustrated features use of the cannulas described herein, the minimally invasive method may be carried out using a different access system including the access devices and systems disclosed in U.S. application Ser. No. 12/209,408, entitled “Multi-Instrument Access Devices and Systems”, filed Sep. 12, 2008, Attorney Docket TRX-1700, which is incorporated herein by reference.

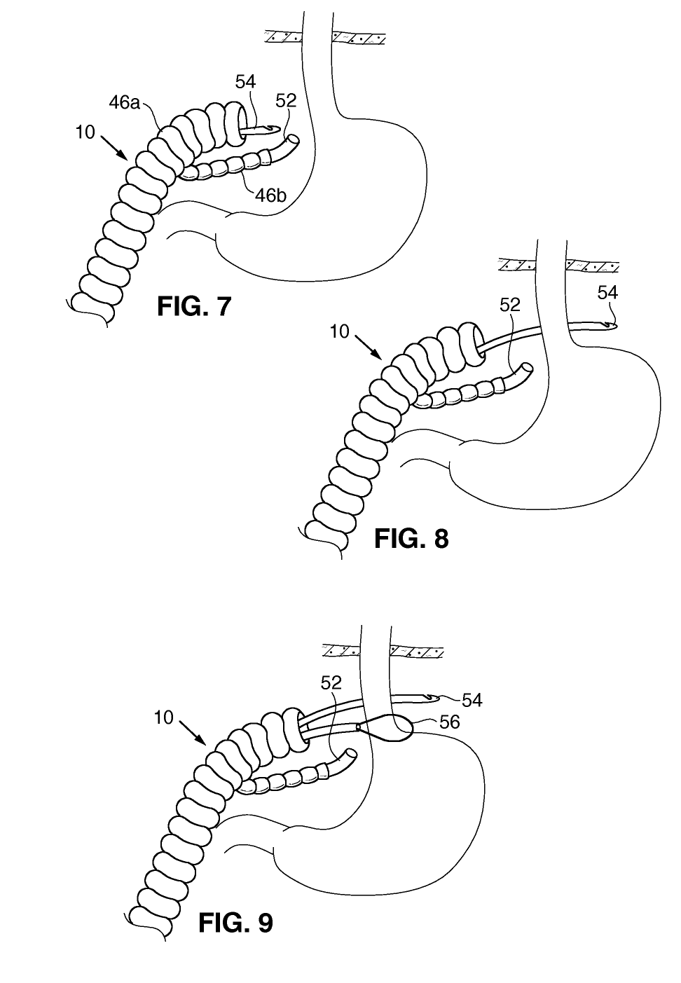

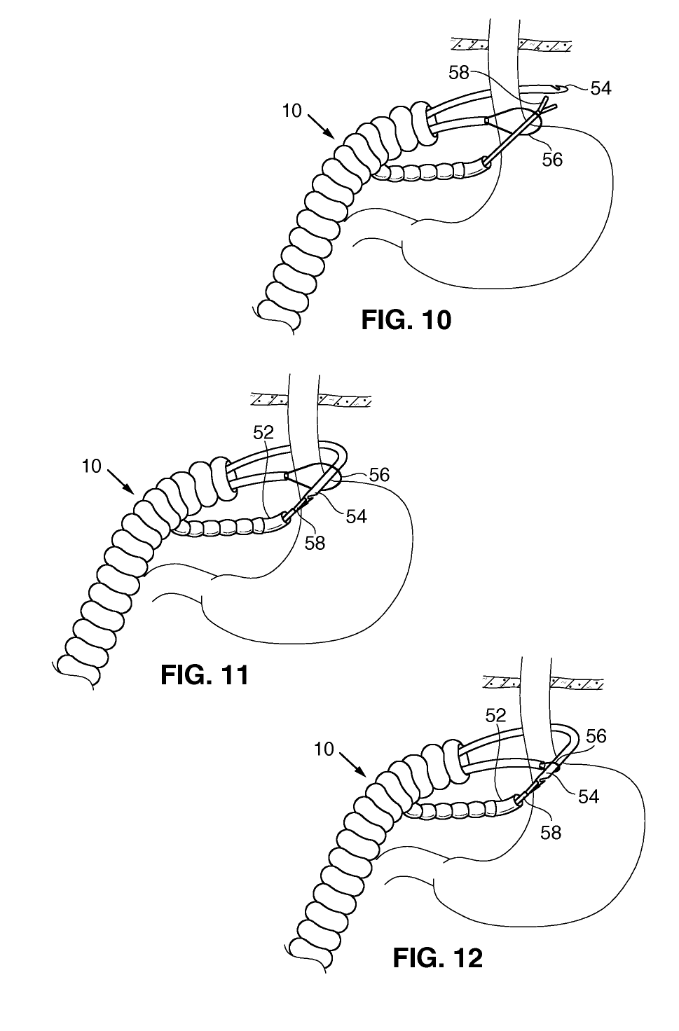

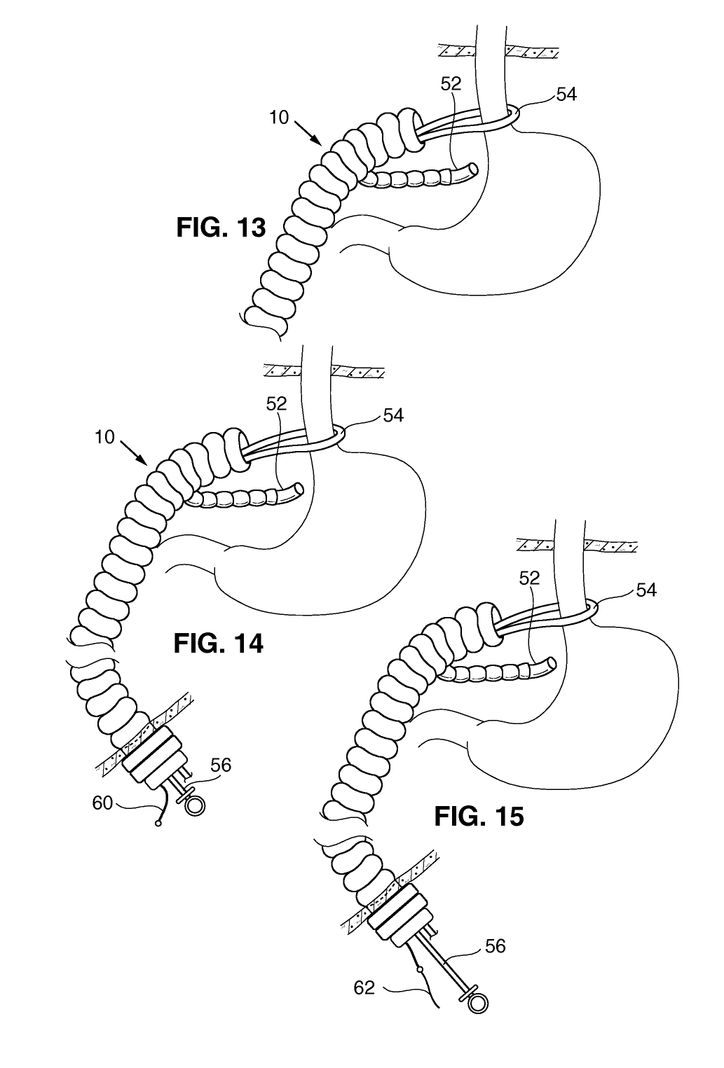

Referring to FIG. 6, cannula 10 is positioned in an incision I or trocar puncture in the abdominal wall, or into an access port giving sealed access to the abdominal cavity. An endoscope 52 is inserted into the cannula, advanced through the side branch 46b and positioned within the abdominal cavity. Under visualization using the endoscope, cannula 10 is deflected using controls 16 as discussed above, until the distal end of cannula 10 is optimally positioned in proximity to the proximal stomach. Next, a blunt dissection instrument 54 is passed through the main branch 46a of the cannula 10 as shown in FIG. 7, and passed posterior to the stomach and/or esophagus as in FIG. 8, forming a tunnel through the fascia/connective tissue surrounding the proximal stomach and lower esophagus. Referring next to FIG. 9, a snare 56 is introduced into the cannula 10 and advanced to a position anterior to the stomach. An endoscopic grasper 58 is passed through an instrument channel in the endoscope 52, advanced through the loop of snare 56, and used to grasp the blunt dissection instrument 54 as shown in FIG. 10. The blunt dissector 54 is withdrawn through the snare 56 using the grasper 58 (FIG. 11), after which the snare 56 is closed around the blunt dissector 54 (FIG. 12) to engage the blunt dissector using the snare. The blunt dissector 54 is released from the jaws of the grasper, and the grasper is withdrawn from the endoscope 52. The snare is withdrawn into the cannula 10, carrying the tip of the blunt dissector 54 into the cannula 10 as shown in FIG. 13.

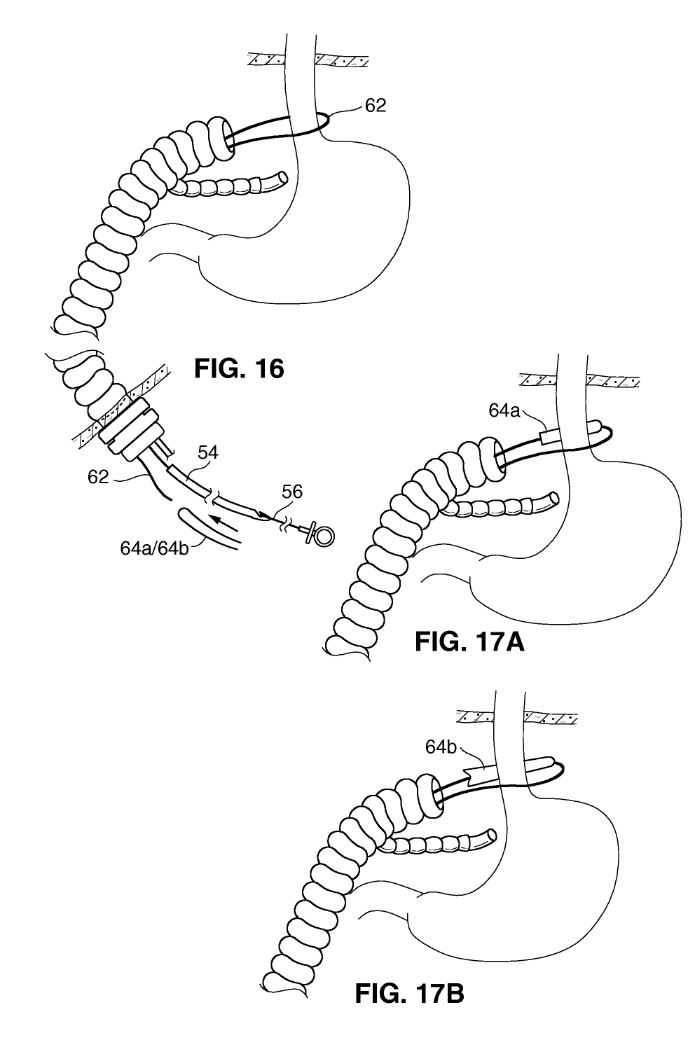

Referring to FIG. 14, at this stage of the procedure the proximal end of the blunt dissector 54, or a tether 60 connected to it, remains outside the body. A guidewire 62 is attached to the proximal end or tether 60 as shown in FIG. 15, and the snare is withdrawn as shown in FIG. 16 to fully withdraw the blunt dissector 54 from the body. Retraction of the blunt dissector 54 carries the distal end of the guidewire with it, such that the guidewire 62 extends through the cannula 10, loops around the lower esophagus or proximal stomach, passes back into the cannula and out of the body. Referring to FIG. 17A, the guidewire 62 preferably includes a balloon dissector 64a or a ribbon dissector 64b on it that is expanded from a collapsed position to an expanded position after it passes from the distal end of the cannula 10 to further dissect the tissue surrounding (and to thus expand) the tunnel originally formed by the blunt dissector 54. Continued passage of the expanded dissector 64a, 64b further dissects the connective tissue surrounding the lower esophagus/proximal stomach as shown in FIGS. 17A and 17B. Dissection may proceed in a number of ways. For example, the guidewire may be pushed or pulled to advance the dissector 64a, 64b through the tissue while maintaining the dissector in its expanded state. Alternatively, an incremental dissection sequence may be performed whereby the guidewire is pushed or pulled with the dissector 64a, 64b in a collapsed state to inch the dissector 64a, 64b forward, and whereby the dissector is expanded between advancing steps to dissect the surrounding tissue, and then collapsed for further advancing of the collapsed dissector further along the tunnel.

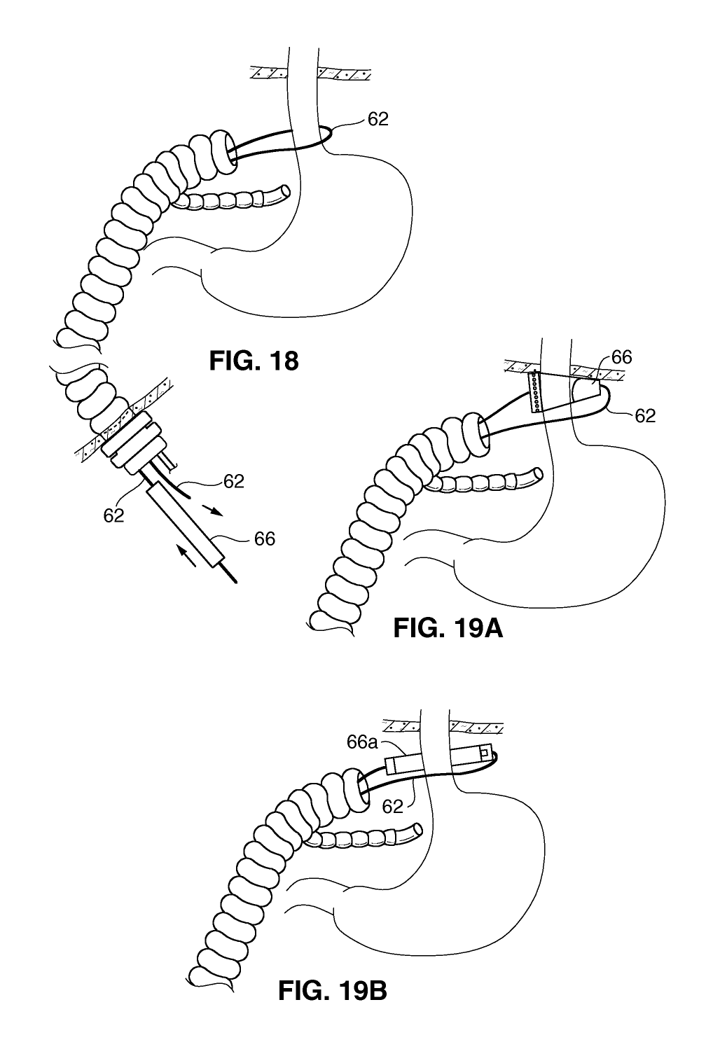

Following dissection, the balloon dissector is withdrawn from the guidewire 62, and an implant 66 is attached to one of the free ends of the guidewire as shown in FIG. 18. The other free end of the guidewire is pulled proximally as shown, carrying the implant 66 into position posterior to the stomach/lower esophagus as shown in FIG. 19A. FIG. 19B illustrates a similar procedure for implantation of a gastric band 66a.

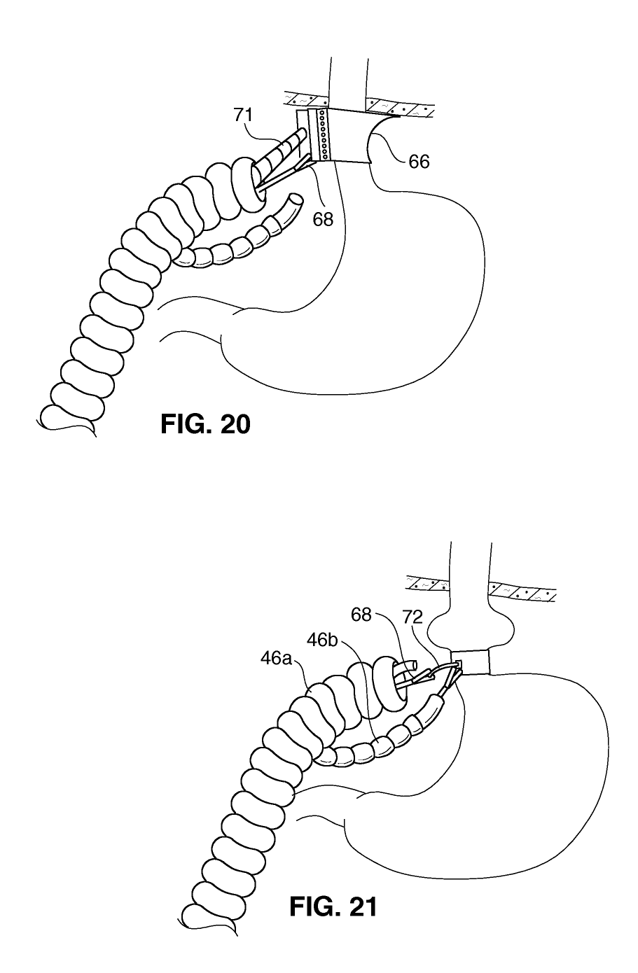

Referring to FIG. 20, if appropriate for the particular implant, grasper 68 is used to fold the implant 66 around the gastro-esophageal junction region as shown such that its free ends overlap. An articulating closure instrument is advanced through the cannula and used to fire fasteners through the overlapping ends of the implant. The guidewire, closure instrument 70 and grasper 68 are removed, leaving the implant in place.

If, rather than being the type of device shown in FIG. 19A, the implant is a gastric band comprising a band lined with an inflatable balloon, once the implant 66a has been drawn to the posterior position shown in FIG. 19B, the endoscope 52 is withdrawn from the side branch 46b and advanced into the body cavity via the main branch 46a. A first pair of graspers 68 is passed through main branch 46a and a second pair 70 is advanced through side branch 46b as shown in FIG. 21. The graspers are used to position and lock the device in place and to couple an inflation tube 72 to a port on the device. The implant is inflated via the inflation tube 72, and the tube 72 is then connected to a small fill port that is placed under the skin of the abdomen for future adjustment of the band diameter.

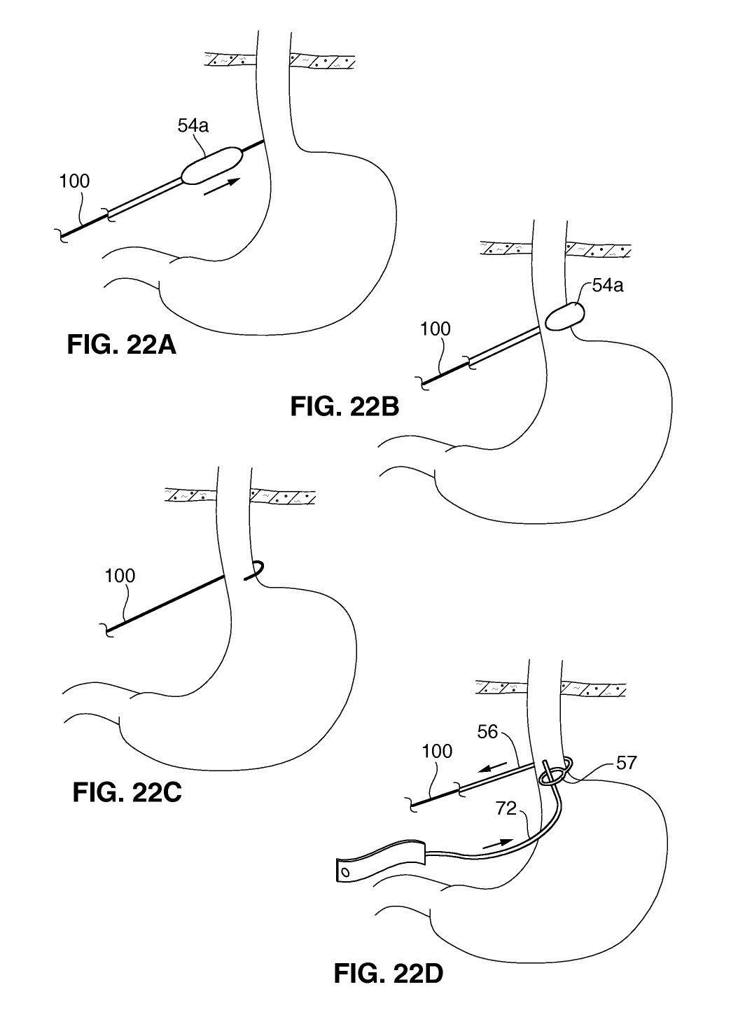

FIGS. 22A-22D disclose an alternate method useful for implanting a gastric banding device. This method may be carried out using the cannulas described herein, or using a different access system including the access devices and systems disclosed in U.S. application Ser. No. 12/209,408, entitled “Multi-Instrument Access Devices And Systems”, filed Sep. 12, 2008, Attorney Docket TRX-1700, which is incorporated herein by reference.

Referring to FIGS. 22A and 22B, a blunt dissector such as balloon dissector 54a is introduced over a guidewire 100 and advanced to dissect a path around the posterior side of the stomach. It should be noted that the tissue undergoing dissection in this and the prior embodiment is not shown for simplicity. The dissector 54a is withdrawn, leaving the guidewire 100 in place (FIG. 22C). Snare 56 is advanced over the guidewire and the snare loop is opened. The inflation tube 72 of the gastric band is advanced through the open snare loop 57. FIG. 22D. The snare loop is closed to engage the tube 72, and tension is applied to the snare to withdraw the snare and the tube 72 around the posterior side of the stomach and then anteriorly. The gastric band is closed using graspers as described above or using alternate techniques.

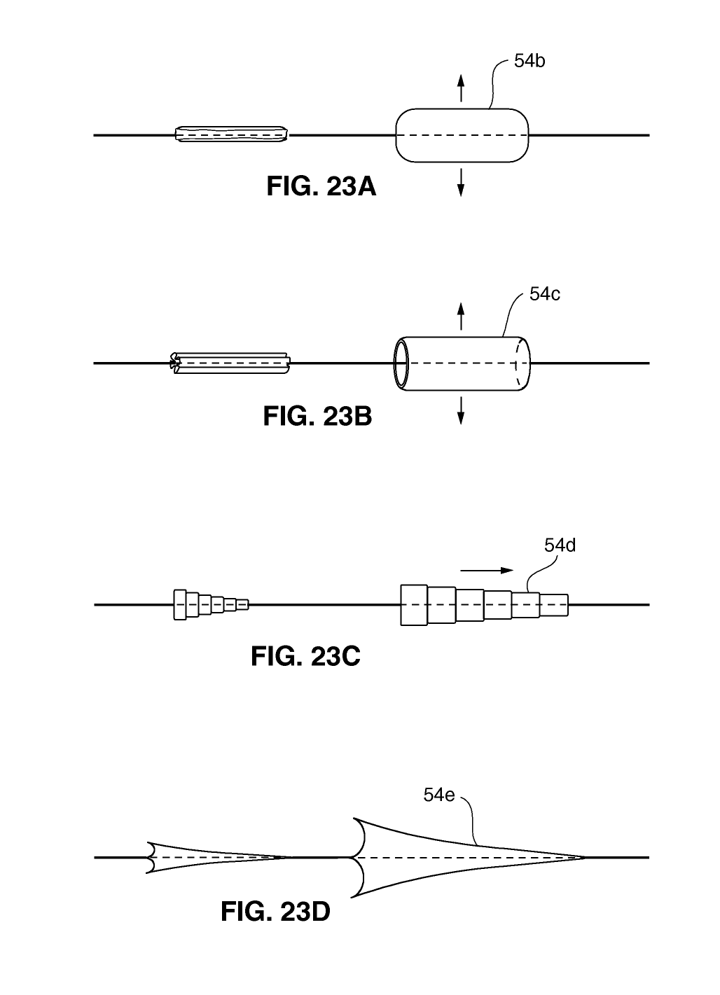

FIGS. 23A through 23D illustrate various embodiments of expandable dissectors 54b-54e, in both collapsed and expanded positions, that may be used for this purpose. The FIG. 22A-22C embodiments illustrate balloon dissectors of varying shapes, each of which may be mounted on a guidewire having an inflation lumen extending through it. Each figure shows the shape of dissector in its expanded position on the right side of the wire, and the shape of the dissector prior to expansion on the left side of the wire.

The FIG. 22D embodiment illustrates a ribbon dissector 54e that may self expend once it passes from the cannula, or that may include a pullwire element extending through a lumen in the guidewire. According to this variation, the pullwire may be withdrawn to expand the ribbon dissector. In alternative embodiments, balloon or ribbon dissectors having similar features may be tracked over the guidewire (e.g. by a separate catheter carrying the dissector) rather than being mounted to the guidewire. Other forms of dissection, including those using laser dissection catheters tracked over the guidewires, are equally suitable for use in the disclosed method.

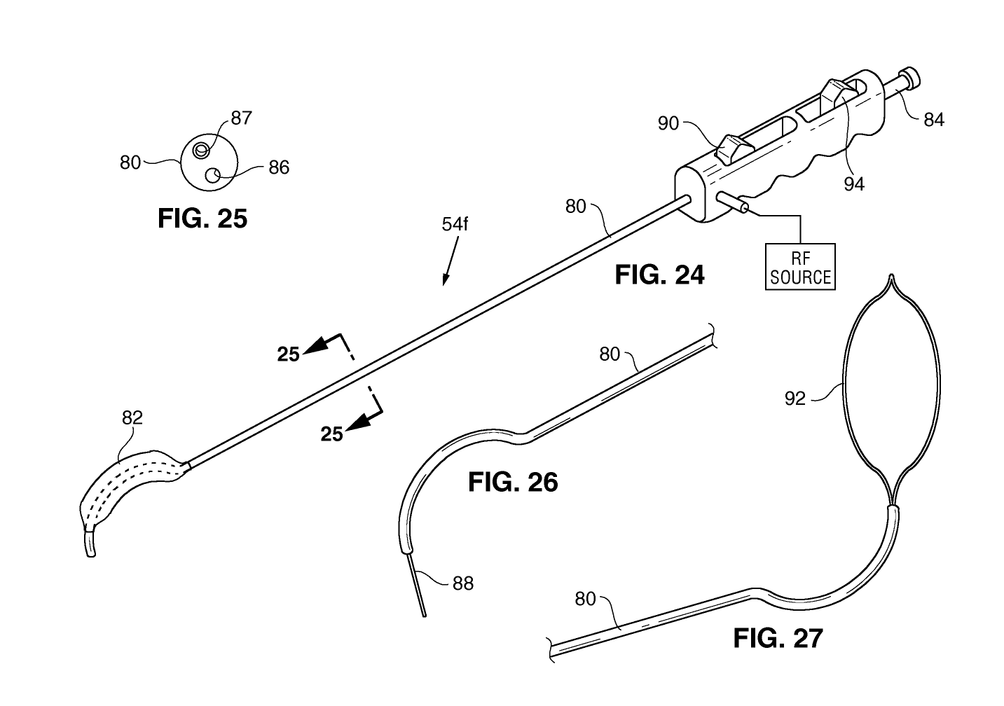

An alternative dissector 54f is shown in FIG. 24. Dissector 54f includes an elongate shaft 80 having a pre-curved distal end. The shaft is preferably rigid or semi-rigid so as to allow it to approximate retain its shape during use, although in alternative embodiment flexible shafts may be used. The curvature of the distal end is selected to cause the dissector to pass posteriorly around the stomach when advanced through the connective tissue. An optional dissection balloon 82 may be positioned on the shaft at the curved section as shown or elsewhere. Balloon 82 is inflatable using inflation medium directed from an inflation port 84 through a lumen 86 fluidly coupled to the balloon.

A monopolar RF dissection wire 88 is positioned within the shaft 80 and has a conductive tip or electrode extendable from the shaft 80 as shown in FIG. 26. The wire 88 is spring loaded in a retracted position, and is advanceable using an actuator such as slider 90 (FIG. 24). The wire 88 is energized using a source of RF energy 90 coupled to the dissector. The dissector 54f allows dissection to be performed using RF dissection, blunt dissection using the balloon or the distal tip of the device, or any combination thereof.

A snare loop 92 is extendable from and retractable into the distal end of the shaft 80 using sliding actuator 94. A lumen 87 in the shaft carries the RF dissection wire and the snare 92.

The dissector 54f of FIG. 24 can be used to simplify the procedure shown in FIGS. 22A-22D by allowing the dissection step (FIGS. 22A and 22B), and the step of engaging the implant (FIG. 22D) to be carried out with a single device. In particular, the device 54f is advanced through an access device into the abdominal cavity, and manipulated using RF and/or blunt dissection to form an appropriate path through the connective tissue. As the device 54f is advanced to the posterior side of the stomach, the curvature of the device carries the distal end of the device into a position appropriate for deployment of the snare (a position similar to that shown in FIG. 22D).

It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the claims and their equivalents.

Any and all applications referred to herein, including for purposes of priority, are hereby incorporated herein by reference.

1-6. (canceled)

7. A dissector for body tissue comprising:
a shaft having a proximal portion and a distal portion;
a dissection element carried by the shaft; and
a snare extendable from the distal portion.

8. The dissector of claim 7, wherein the dissection element includes an expandable balloon disposed on the shaft.

9. The dissector of claim 7, wherein the dissection element includes a conductive element, the conductive element electrically connectable to a source of radiofrequency energy.

10. The dissector of claim 9, wherein the dissection element further includes an expandable balloon disposed on the shaft.

11. A method for positioning a band around a stomach in a body cavity, the method comprising the steps of:
forming a percutaneous incision and positioning an access device within the incision for access through;
placing the band within the body cavity, the band having first and second end portions;
introducing a dissector through the access device into the body cavity, and using the dissector to form a tunnel around a posterior side of the stomach;
introducing a snare through the access device into the body cavity, and advancing the snare through the tunnel;
engaging the first end portion of the band with the snare, and withdrawing the snare through the tunnel to position the band around the stomach; and
using instruments passed through the access device, coupling the first and second end portions to close the band.

12. The method of claim 11, wherein introducing the dissector includes passing the dissector over a guidewire, wherein the method includes withdrawing the dissector from the guidewire, and wherein introducing the snare includes passing the snare over the guidewire.

13. The method of claim 11, wherein introducing the snare includes extending the snare from a distal portion of the dissector.

14. The method of claim 11, wherein introducing a dissector includes introducing a dissector having a pre-curved distal end, and orienting the dissector such that the pre-curved distal end curves around the posterior side of the stomach.

15. The method of claim 11, wherein using the dissector to form a tunnel includes using a conductor on the dissector to deliver ablative energy to the tissue.

16. The method of claim 11, wherein using the dissector to form a tunnel includes expanding a dissection balloon on the dissector.

17. The dissector of claim 7, wherein the conductive element is extendable from and retractable into the distal portion of the shaft.

18. The dissector of claim 17, wherein the shaft includes a lumen, and wherein the conductive element is extendable from and retractable into the lumen.

19. The dissector of claim 18, wherein the snare is extendable from and retractable into a lumen in the shaft.

20. The dissector of claim 7, wherein the snare is extendable from and retractable into a lumen in the shaft.

21. The dissector of claim 7, wherein the shaft has a precurved distal end.

22. The method of claim 11, including introducing an elongate shaft through the access device into the body cavity, energizing a conductive element disposed on a distal portion of the elongate shaft to form the tunnel, and extending the snare from a distal portion of the shaft.

23. The method of claim 22, further including expanding a balloon on the shaft and advancing the expanded balloon through the tunnel formed using the conductive element.
